(19) United States
(12) Patent Application Publication
(10) Pub. No.: US 2004/0029774 A1
(43) Pub. Date: Feb. 12, 2004

(54) Composition and methods for the treatment of musculoskeletal disorders and collagen and elastin deficiencies

(76) Inventor: Aly Gamay, McLean, VA (US)

Correspondence Address: Womble Carlyle Sandridge & Rice, PLLC, P.O. Box 7037, Atlanta, GA, 30357-0037, US

(21) Appl. No.: 10/213,057

(22) Filed: Aug. 6, 2002

(51) Int. Cl. A61K 38/39; A61K 31/7008; A61K 9/14

(52) U.S. Cl. 514/2; 514/62; 424/488

(57) ABSTRACT

Disclosed is a composition and method of enhanced nutrients delivery system for the treatment of musculoskeletal disorders and promotion of collagen and elastin synthesis in mammals by the oral administration of gel-like composition of hydrated Chondoritin, Glucosamine, MSM (Methyl-Sulfonyl-Methane), gelatin, hydrolyzed gelatin, collagen, and/or hydrolyzed collagen in combination with gelling agents. The increased bioavailability of the composition aids in the relief of joint pain and rebuilds cartilages, tendons, muscles, skin and connective tissues.

### FIELD OF INVENTION

The present invention relates to a composition for treating musculoskeletal disorders and collagen and elastin deficiencies and in particular to the administration of a gel-like composition having increased bioavailability.

### BACKGROUND

Damage to the collagen and elastin containing tissues of the body can lead to sever malfunction of organs and cause serious disorders. The damage can be attributed to a variety of factors such as aging, poor nutrition, diseases and physical trauma. It is important from a therapeutic point of view to conserve intact tissues and to assist in the rapid repair of damaged tissue. Proper nutrition and ingestion of rejuvenating-nutrients can help in maintaining healthy tissues.

Most cells in multicellular organisms are in contact with an intricate meshwork of interacting, extracellular macromolecules that constitute the extracellular matrix. These versatile protein and polysaccharide molecules are secreted locally and assemble into an organized meshwork in the extracellular space of most tissues. In addition to serving as a type of universal biological glue, they also form highly specialized structures such as cartilage, tendons, basal laminae, and bone and teeth.

Until recently, the vertebrate extracellular matrix was thought to serve mainly as a relatively inert scaffolding that stabilized the physical structure of tissues. But now it is clear that the matrix plays a far more active and complex role in regulating the behavior of the cells that contact it—influencing their development, migration, proliferation, shape, and metabolic functions.

Local cells, especially fibroblasts, which are widely distributed in the matrix, secrete the macromolecules that constitute the extracellular matrix. Two of the main classes of extracellular macromolecules that make up the matrix are (1) the collagens and (2) the polysaccharide glycosaminoglycans, which are usually covalently linked to protein to form proteoglycans. The glycosaminoglycan and proteoglycan molecules form a highly hydrated, gel-like “ground substance” in which collagen fibers are embedded. While the long collagen fibers strengthen and help to organize the matrix, the aqueous phase of the polysaccharide gel permits the diffusion of nutrients, metabolites, and hormones between the blood and the tissue cells. In many cases, fibers of the rubber-like protein elastin are also present and impart resilience to the matrix. In addition, two high molecular weight glycoproteins are among the major components of extracellular matrices: fibronectin, which is widely distributed in connective tissues, and laminin, which has so far been found only in basal laminae.

The term connective tissue is often used to describe the extracellular matrix plus the cells found in it, such as fibroblasts, macrophages, and mast cells. The amount of connective tissue in organs varies greatly: the cornea, skin and bone are composed mainly of connective tissue, whereas the brain and spinal cord contain very little. Moreover, the relative amounts of the different types of matrix macromolecules and the way that they are organized within the extracellular matrix vary enormously, giving rise to a diversity of forms, each highly adapted to the functional requirements of the particular tissue. Thus, the matrix can become calcified to form the rock-hard structures of bone or teeth, it may take on the rope-like organization of the collagen fibers in tendons, which gives them their enormous tensile strength, or it can form a clear window-like structure in the cornea.

Arthritis, a musculoskeletal disorder, is the leading cause of disability in the United States. The Centers for Disease Control and Prevention (CDC) stated that arthritis and other rheumatic conditions accounted for about 744,000 hospitalizations and 4 million days of care in 1997. Forty million Americans, representing 15% of the population, have some form of arthritis, and that figure is expected to increase to 59.4 million (18.2%) by the year 2020, an increase of 57% in the number of persons affected. Arthritis patients make more than 315 million physician visits and are hospitalized more than 8 million times a year. Arthritis costs the nation $65 billion annually in medical costs and lost productivity. Osteoarthritis (OA), or degenerative joint disease, is the most common type of arthritis, affecting 20.7 million (12.1%) of U.S. adults in 1990, now estimated at 37 million, and trailed chronic heart disease as the leading cause of Social Security payments due to long-term absence from work. Lawrence R C, et al. Arthritis & Rheumatism 1998; 41:778-799.

Osteoarthritis usually is present as pain, which worsens with exercise or simply regular activities. X-ray can clearly show the status of the thinning cartilage. Common joints affected are the knees, hips and spine, finger, base of thumb and base of the big toe. Osteoarthritis is characterized by degenerative changes in the articular cartilage and subsequent new bone formation at the articular margins. The primary defect in hyaline cartilage, at the articular surface of the joint, is an alteration in the ratio of total glycosaminoglycans to that of the collagen fiber content in the matrix. Yasuda K. Hokkaido Igaku Zasshi July 1997; 72(4): 369-76. Paleontologists have found osteoarthritis to exist in almost every vertebrate, Tindall W N. Business & Health December 1997, 47-48. Bones directly underneath the cartilage in joints are called subchondral bones. This bone nourishes the cartilage with oxygen, water, and nutrients conveyed through microscopic channels. This supply route carries “chondroprotective agents” from the bloodstream to the cartilage.

There is no definitive answer regarding the cause of osteoarthritis. A natural erosion of cartilage occurs with age, but excessive loads placed on joints, obesity, heredity, trauma, decreased circulation, poor bone alignment, and repetitive stress motion play a role. Osteoarthritis may also be the result of free radical damage, thought to be a major cause of many diseases, including the aging process, cancer, heart disease and degenerative diseases.

There is no known drug that claims to reverse osteoarthritis. Most therapeutic agents are directed at reducing the inflammation and relieving pain. Non-steroidal anti-inflammatory drugs (NSAIDs) are the first line of treatment for osteoarthritis, but long-term use can lead to gastric ulcers, kidney damage, and hearing loss and even inhibit cartilage formation.

Most western countries have adopted traditional western medicine to treat bone and joint inflammation. The treatments usually involve synthetic drugs, such as Motrin, Fildene, Indocin, Clinoril, Naprosyn, Vicoden, and Meclomen. These drugs do not always alleviate pain and discomfort, or restore significant use of inflamed joints. Moreover, such drugs may lead to undesirable side effects. Thus, there is a need for a treatment that does not include such drawbacks as described above.

### SUMMARY

The present invention comprises both a composition and method for treating musculoskeletal disorders and collagen and elastin deficiencies. The composition is typically taken orally by a human or animal as a dietary supplement. The dietary supplement provides both collagen and elastin building nutrients in a bioavailable format. Furthermore, the composition can increase the synthesis of collagen and cartilage mass to compensate for the loss of cartilage.

The composition includes a dietary supplement for treating an animal. The composition includes a gelling agent and an additive selected from the group consisting of glucosamine, chondroitin, methyl-sulfonyl-methane, gelatin, hydrolyzed gelatin, collagen, hydrolyzed collagen and mixtures thereof. The gelling agent may be selected from the group of gelling agents including collagen, gellan gum, carbohydrate gel forming polymers, carrageenan, alginates, guar, xanthan, carboxymethyl cellulose, starch and mixtures thereof. Furthermore the gelling agent or composition may be hydrolyzed. The composition comprises between about 15% to about 70% water. The composition may further include an acidulant such that the composition has a pH of between about 2.5 to about 5.5.

The method of the present invention includes making a composition for treating a human or animal. The method includes providing a gelling agent and providing an additive selected from the group consisting of glucosamine, chondroitin, methyl-sulfonyl-methane, gelatin, hydrolyzed gelatin, collagen, hydrolyzed collagen and mixtures thereof. The method also includes combining the gelling agent and additive to form the composition. Additionally, the method includes hydrating the composition, wherein water heated to a temperature of between about 110° F. to about 170° F. can be added. The method further can include pasteurizing the composition.

A further embodiment includes a method of treating a human or animal for musculoskeletal disorders and collagen and elastin deficiencies. The method includes administering to a human or animal a composition comprising a gelling agent and an additive selected from the group consisting of glucosamine, chondroitin, methyl-sulfonyl-methane, gelatin, hydrolyzed gelatin, collagen, hydrolyzed collagen and mixtures thereof. The gelling agent may be selected from the group consisting of collagen, gellan gum, carbohydrate gel forming polymers, carrageenan, alginates, guar, xanthan, carboxymethyl cellulose, starch and mixtures thereof. Furthermore, the glucosamine is administered in a dosage of about 200 mg to about 2000 mg and the chondroitin is administered at a dosage of 100 mg to about 2000 mg. Additionally, the methyl-sulfonyl-methane is administered at a dosage of 100 mg to about 2000 mg.

A further embodiment includes a kit comprising a preformed package. The kit includes a gelling agent and an additive selected from the group consisting of glucosamine, chondroitin, methyl-sulfonyl-methane and mixtures thereof. The kit additionally includes the preformed package which is hermetically sealed. The composition may be a gelled food.

### DETAILED DESCRIPTION

The present composition includes a dietary supplement for treating an animal or human. The composition includes a gelling agent and an additive selected from the group consisting of glucosamine, chondroitin, methyl-sulfonyl-methane gelatin, hydrolyzed gelatin, collagen, hydrolyzed collagen and mixtures thereof. The gelling agent may be selected from the group of gelling agents including collagen, gellan gum, carbohydrate gel forming polymers, carrageenan, alginates, guar, xanthan, carboxymethyl cellulose, starch and mixtures thereof.

The present method includes the formation of a composition for treating a human or animal. The method includes providing a gelling agent and providing an additive selected from the group consisting of glucosamine, chondroitin, methyl-sulfonyl-methane gelatin, hydrolyzed gelatin, collagen, hydrolyzed collagen and mixtures thereof. The method also includes combining the gelling agent and additive to form the composition. Additionally, the method includes hydrating the composition, wherein water heated to a temperature of between about 110° F. to about 170° F. can be added. The method further can include pasteurizing the composition.

The components may be formulated for administration into one composition containing all the components. Alternatively, the components may be formulated into more than one composition, each of which contains one or more components. In addition, each component may constitute a separate composition, and be administered separately in conjunction with a gelling agent.

The components may be mixed in any order to prepare the composition. The composition may comprise the same components that were added to the mixture, or any components that result from an interaction between two or more of the components after mixing.

All conditions characterized by joint, tendon and connective tissues inflammation and degeneration (all forms of arthritis, rheumatism, including rheumatoid arthritis, bursitis, tendonitis, gout, etc.), weak muscles, dry skin, skin wrinkles and all disorders related to collagen and elastin defects are able to benefit from the composition of the invention. The composition is effective for all mammals, including farm animals, laboratory animals, pet animals, and humans. The terms rejuvenating-nutrients and/or selected additives refer to the nutrients for treating conditions previously mentioned.

The components of the composition are in a form that is systemically absorbable in an animal or human. The components can be pre-hydrated or pre-solubilized either individually or in various combinations for enhanced bioavailability. After being absorbed, the components of the composition, or their metabolic products, are delivered to the inflamed cartilages, joints or connective tissues or organs.

The present rejuvenating-nutrient fortified food products may be produced in various formats such as semi-liquid drinkable snacks, semi-liquid preparation that could be mixed with water or other fluids, gelled-dessert type food, powder blend that is hydrated in water before consumption or as soft or hard gel products. In an embodiment, further it essentially comprises sufficient amounts of a gelling agent to provide the finished products with a gel strength at the desired moisture levels herein of about 20-50%.

The gelled structure essentially entraps various rejuvenating-nutrients in hydrated status available for fast disintegration and enhanced absorption in the small intestine. The gel-like structure maintains the present rejuvenating-nutrients in a hydrated status, ready for ingestion without the need for fluids either to aid in swallowing or allow solubilization of rejuvenating-nutrients.

The gelled structure further allows for packing high concentrations of various rejuvenating-nutrients in relatively small volume. The gelling agents aid in masking off-flavors and unpleasant taste and allows for incorporation of flavors and colors. Furthermore, the gelled structure enhances the packaging and portability of finished products.

The soft texture of the gelled structure allows for ease of consumption such that it can be a chewable snack. Furthermore, the gel matrix melts at about 95° F. to 100° F., which is close to the temperature range of the human body. This temperature range allows for the melt down of the gel matrix and immediate release of rejuvenating-nutrients once the product is consumed.

Gelling Agent

The present gelling agents are to be indistinguishable from mere thickening agents as to the way they are referred to herein. The gelling agent may be used as a thickening agent at lower concentration. On the other hand, a thickening agent in combination with another ingredient may produce a set consistency. Good results are obtained when all or at least a portion of the gelling agent is supplied by a member from the group consisting of collagen (gelatin), gellan gum, carbohydrate gel forming polymers (such as pectin, gel forming starches, dextran, agar, and mixtures thereof), carrageenan, and alginates. Other thickening agents may include but not be limited to guar, Xanthan, Caribbean gums, Carboxymethyl cellulose and thickening starches like rice, potato, and tapioca starches. Gelling agent that forms an irreversible gel may also be utilized. An irreversible gel is a gel that will set quickly, but will also tend to degrade in texture and strength under conditions of increased shear and temperature.

The particular gelling agent(s) usage level depends upon a variety of factors such as the desired textural properties in the finished product, total solids level and type, and strength of the gelling agents. Generally, however, good results are obtained when the total gelling agent is present at levels ranging from about 5% to 20%.

An example gelling agent includes gelatin or gel forming starches or combinations of both. Gelatin serves as a gelling agent as well as one of the nutrients that help in the treatment of several musculoskeletal disorders. Gelatin is derived from denatured collagen.

Collagens are a family of highly characteristic fibrous proteins found in all multicellular animals and may serve as a gelling agent. They are the most abundant proteins in mammals, constituting 25% of their total protein. The central feature of all collagen molecules is their stiff, triple-stranded helical structure. Three collagen polypeptide chains, called alpha-chains, are wound around each other in a regular helix to generate a rope-like collagen molecule about 300 nm long and 1.5 nm in diameter. At most, eleven genetically distinct collagen types have been well defined. The major types are referred to as types I, II, III, IV and V. Types I, II and III are the main types of collagen found in connective tissues, and of these, type I is the most common, constituting about 75% of the collagen in the body. After being secreted into the extracellular space, types I, II and III collagen molecules assemble into ordered polymers called collagen fibrils, which are long (up to many microns), thin (10 to 300 nm in diameter), cable-like structures clearly visible in electron micrographs. Such fibrils are often grouped into larger bundles, which can be seen in the light microscope as collagen fibers several microns in diameter.

Tissue such as skin requires elasticity in addition to tensile strength in order to function. An extensive network of elastic fibers in the extracellular matrix of these tissues gives them the required ability to recoil after transient stretch. The main component of elastic fibers is elastin, a 70,000-dalton glycoprotein, which, like collagen, is unusually rich in proline and glycine but, unlike collagen, contains little hydroxyproline and no hydroxylysine.

Collagen is the main constituent of the supporting tissue and connective tissue in animals and humans and, more particularly, is found in the skin, the tendons and bones. Collagen may be produced from animal skin, animal bones and other sufficiently purified connective tissue. Gelatin is a denatured collagen, which is soluble only in hot water and upon cooling is capable of binding considerable amounts of water.

The collagen fibers are responsible for the solidity of the dermis. These are very resistant but sensitive to certain enzymes generally deemed collagenases. In the dermis, the collagen fibers consist of fibrils firmly attached to each other, thus forming more than ten types of different structures. The structure of the dermis is in large part due to the entanglement of the packed collagen fibers. The collagen fibers participate in the tonicity of the skin.

Gelatin is obtained by the controlled hydrolysis from fibrous insoluble collagen. The pre-hydrated gelatin is suitable for immediate ingestion and digestion, and absorbable in the digestive tract. Being a protein, gelatin is composed of a unique sequence of amino acids. The characteristic feature of gelatin is the high content of amino acids Glycine, Proline and Hydroxyproline. Structurally, gelatin molecules contain repeating sequences of Glycine-X-Y triplets, where X and Y are frequently Proline and Hydroxyproline. These sequences are responsible for the triple helical structure of gelatin and its ability to form gels where helical regions form in the gelatin protein chains immobilizing water of hydration. The amino acid composition of gelatin is: Glycine 27%, Proline and Hydroxyproline 25%, Glutamic acid 10%, Alanine 9%, Aspartic acid 6% and other amino acids 15%. The raw materials, type of pre-treatment and gelatin extraction conditions all affect the molecular distribution of the gelatin polypeptides. Commercial gelatins are heterogeneous protein mixture of polypeptide chains. Gelatin molecules are quite large with molecular weight ranging from a few thousands up to several hundred thousands daltons. The molecular weight distribution of gelatin has a great bearing on the physical properties of gelatin and particularly affects viscosity and gel strength.

A gel forming (or thickening) starch can be used alone or in combination with gelatin as a supplemental gelling ingredient. The starch gelling ingredient may be present at about 1% to 20% in addition to gelatin content ranging from about 1% to 15%. An example of a suitable starch is available from Flojel 60 from National Starch and Chemical Company.

Rejuvenating Additives:

Glucosamine is an amino sugar normally found in humans and derived from glucose. It is considered the starting point for the synthesis of macromolecules such as glycoproteins, glycolipids, and glyco-aminoglycans or mucopolysaccharides. Glycosamine is one of the biological chemicals involved in the formation of cushioning ingredients for joint fluids and surrounding tissues and contributes to make synovial fluid thick and elastic in joints and vertebrae.

Glucosamine, which is naturally found in high concentrations in joint structures, is a stable, tasteless and water-soluble nutrient. It is readily absorbed from the intestines, stays in the blood for several hours, and very little is excreted. In particular, glucosamine stimulates the body's manufacture of collagen, the protein portion of the fibrous substance that holds joints together.

Glucosamine from exogenous sources (food and supplements) may stop the progression of cartilage degradation and stimulate the production of new cartilage. Glucosamine absorbed by the gastrointestinal tract undergoes significant first-pass metabolism in the liver, with the resulting 26% bioavailability. It is incorporated into plasma proteins as a result of hepatic metabolism, and concentrates in the articular cartilage. Clinical improvement of symptoms has been seen as early as one week after oral administration of glucosamine sulfate and has persisted for up to four weeks after discontinuation. Barclay T S, Tsourounis C, McCart G M. Glucosamine. Annals of Pharmacotherapy 1998; 32:574-79.

Several commercial forms of glucosamine are available, including the sulfate, hydrochloride, and N-acetylglucosamine (NAG). Glucosamine hydrochloride has a higher concentration of glucosamine than the sulfate form. NAG is rapidly metabolized to make proteins and provides less glucosamine for cartilage repair. The composition of the invention could include one or a combination of the glucosamine forms. The dosage range for glucosamine can vary from 500 mg to 3000 mg a day, in divided doses, depending on body weight and severity of symptoms. D-Glucosamine HCL (99%) may be used at 200 to 2000 mg per dosage.

Chondroitin sulfate is the major glycosaminoglycan in cartilage and may have a synergistic effect with glucosamine. Chondroitin is not easily absorbed by the body as an oral dosage. Chondroitin sulfate is half galactosamine, which may be made directly from glucosamine and has great water retaining ability. Chondroitin Sulfate (90%) may be added at 100 to 2000 mg per dosage.

MSM (Methyl-Sulfonyl-Methane) is also known as dimethyl sulfone or sulfonylbismethan. MSM (99%) may be utilized at 100 to 2000 mg per dosage.

An example composition may include D-Glucosamine HCL (99%) added at 200 to 2000 mg, Chondroitin Sulfate (90%) added at 100 to 2000 mg, MSM (99%) added at 100 to 2000 mg, commercial gelatin may be added at 0.5-22% and hydrolyzed Collagen which is available from commercial sources may be added at 0.5 to 10%. In one embodiment, the amount of various nutrients per serving (10 to 30 g) is varied. For instance, D-Glucosamine HCL is used at 1000 to 2000 mg, Chondroitin Sulfate at 800 to 2000 mg, MSM may be utilized at 100 to 1500 mg, commercial gelatin may be added at 0.5-20% and hydrolyzed Collagen may be added at 0.5 to 10%.

Sweeteners

Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Example nutritive carbohydrate sweetening agents include those selected from the group consisting of sucrose, glucose, fructose, and corn syrup solids.

Non-sugar nutritive sweeteners may be utilized. Examples of non-sugar nutritive sweeteners include mannitol, sorbitol, maltitol, xylitol, lactitol monohydrate, erythritol, and glycerin.

Suitable non-nutritive sweeteners may also be used for sugar-free fictional foods. Example of non-nutritive sweeteners includes Sucralose, Aspartame, Saccharin and other high potency sweeteners. Of course, mixtures of the above-noted materials are contemplated herein.

Flavors

Optionally, the present gel food products can further additionally include effective amounts of flavor(s). If present, such flavors can comprise effective amounts of flavors to provide desired flavor levels. Generally, flavors present at from about 0.01% to about 3% of the finished products are contemplated.

Acidulant

Sufficient amounts of an edible organic acid or acidulant are added to the gel to produce a pH of about 2.5 to 5.5, or about 3.2 to 4.5, and/or results about 3.2 to 3.6. The particular pH selected from within this pH range depends in part upon the type of gelling ingredient employed as well as the organoleptic attributes desired. For example, in the embodiment that contains high levels of joint nutrients and wherein the gelling agent is starch, the pH range may vary from about 3.0 to about 3.6.

A variety of edible organic acids can be used to adjust the pH of the present invention as well as to control the taste and tartness of the present products. For example, citric acid, tartaric acid, malic acid, ascorbic acid, lactic acid, phosphoric acid, hydrochloric acid and mixtures thereof may be used.

Acidulants further provide optimum pH for extended shelf life of functional foods without refrigeration. Furthermore, acidulants enhance flavor perception to affect various flavor profiles.

The acidulants modify the gelled product's pH to a range of about 3.0 to 3.5, which is close to the pH of the human stomach. It is believed that that pH resemblance may accelerate the disintegration of the gelled product and release the desirable nutrients into a closely acidified environment for optimal digestion and absorption in the intestinal tract.

Additional Ingredients

The present food compositions can optionally contain a variety of additional ingredients suitable for rendering such products more organoleptically acceptable, more nutritious and/or more storage stable. Such optional components include fiber materials, colors, coloring agents, vitamins, antioxidants, preservatives emulsifiers, dairy products and animal products.

In one of the embodiments, rejuvenating-nutrients: D-Glucosamine HCL (99%), Chondroitin Sulfate (90%), MSM (Methyl-Sulfonyl-Methane) (99%), Gelatin and/or Hydrolyzed Collagen are added to the rest of ingredients in a processor and heat treated to 165° F. to dissolve all ingredients and affect pasteurization of the gelled product.

To aid in full solubilization and hydration of desirable joint-ingredients, a prehydrating step is employed. D-Glucosamine HCL, Chondroitin Sulfate, MSM, Hydrolyzed Collagen and Gelatin are pre-hydrated either individually or combined. Water may be heated to 110° F-170° F., and then rejuvenating-nutrients are added slowly with agitation to ensure complete hydration and solubilization of rejuvenating-nutrients. The prehydration step may be accomplished over about 10-50 minutes until no visible undissolved particle is observed. In the case of collagen and chondoritin, a viscous semi-liquid intermediate produced is obtained. In case of MSM and Glucosamine, a cloudy liquid is obtained.

Any of food processing vessels may be used to combine and heat-treat the jelled product ingredients. A Lemitech, direct steam injection coprocessor was utilized to mix and pasteurize the gelled products.

Any sequence of ingredients addition may be adopted. In one embodiment, sweeteners are added first to the processor. Next gel forming starch is added with continuous agitation. Heating is commenced to about 160° F.-190° F. Prehydrated joint-ingredients, flavors, acidulants, color are then added and heated to about 150°-160° F.

The resultant pasteurized product has a flowable consistency suitable for further filling into suitable containers. Various filling temperatures between about 100° F.-170° F. may be employed without impacting the product integrity. Cooling of the finished gelled product is optional.

The gelled functional food may be filled using any of the filling equipment known to those skilled in the art of packaging technology. The gelled functional food may be dispensed in trays with cavities of the desired shape and weight, or may be filled into plastic, glass, and synthetic material, paper or like containers or packages.

A soft collagen-support product may be produced in a thick consistency and packaged in squeeze type packages.

Alternately, the collagen and elastin support ingredients may be mixed and sold as a dry blend. Upon using the products, the blend may be added to boiling water, boiled for about 3-5 minutes, dispensed into suitable containers and cooled. The resultant products may be served as a gelled-type dessert.

The gelled functional food may be additionally dispensed into hermetically sealed packages for extended shelf life.

In the case of pet's rejuvenating-nutrients, sweet or salty type of products may be produced. In the case of salty product, no sweeteners are used and savory flavors, i.e. meat and dairy, are added.

The gelled products may be handled and distributed either at room temperature, refrigerated or frozen.

### EXAMPLES

To evaluate the dissociation and solubility of available technology and joints products in the marketplace, a comparative study was initiated. Deionized water was heated to 98° F.-99° F. to mimic the temperature of the human body. Adjustment of pH of the water to 2.7-2.9 was accomplished using hydrochloric acid to effect acidic conditions of the human stomach. One part of studied joints products was added to one part of the aforementioned water in a beaker to approximate the moisture condition of the stomach under a given circumstance. Water/joints products were placed in a heated water bath with continuous agitation. Disintegration of various joints products was compared at 30 minutes, one hour, two hours, and three hours.

The following data reflect the amount of disintegrated product compared to added amount at 0 minutes:

| JOINTS | % DISINTEGRATION | | | |
|---|---|---|---|---|
| PRODUCT | 30 MIN. | 1 HR. | 2 HRS. | 3 HRS. |
| FRUIT CHEWS | 30 | 60 | 80 | 95 |
| CHOCOLATE CHEWS | 30 | 60 | 80 | 95 |
| CHEWABLE WAFERS | 30 | 60 | 80 | 95 |
| POWDER BRAND (1) | 90 | 95 | 95 | 95 |
| PILLS BRAND (1) | 5 | 10 | 20 | 25 |
| PILLS BRAND (2) | 5 | 10 | 20 | 25 |
| PILLS FOR DOGS | 20 | 40 | 45 | 45 |
| PILLS FOR CATS | 20 | 40 | 45 | 45 |
| PRESENT INVENTION (1) | 45 | 95 | 98 | 98 |
| PRESENT INVENTION (2) | 45 | 95 | 98 | 98 |

Since the average residence time of food in the stomach is two to three hours, most of the ingested dose of the pills and tablets will not be utilized in the body.

In order to determine the amount of rejuvenating-nutrient that may be completely dissolved in water under conditions similar to that of stomach, the following study was executed: Deionized water was heated to 98° F.-99° F. to mimic the temperature of the human body. Adjustment of pH of the water to 2.7-2.9 was accomplished using hydrochloric acid to effect acidic conditions of the human stomach. Joints products were added to the aforementioned water in a beaker at 10, 30, and 50% concentrations (in case of chondroitin and hydrolyzed gelatin only, 70% concentration was evaluated because they were 100% soluble at 50% concentration). Water/joints products were placed in a heated water bath with continuous agitation. Solubility of various joints products was compared at 30 minutes, one hour and two hours.

The following data reflect the percentage of product solubilized compared to added concentration at 0 minutes:

| REJUVENATING | CONCEN- | % SOLUBILITY | | |
|---|---|---|---|---|
| INGREDIENTS | TRATION | 30 MINS. | 1 HR. | 2 HRS. |
| MSM | 10% | 100 | | |
| | 30% | 100 | | |
| | 50% | 10 | 20 | 30 |
| GLUCOSAMINE | 10% | 100 | | |
| | 30% | 30 | 30 | 30 |
| | 50% | 10 | 20 | 30 |
| CHONDROITIN | 10% | 100 | | |
| SULFATE | 30% | 100 | | |
| | 50% | 100 | | |
| | 70% | 20 | 20 | 20 |
| GELATIN | 10% | 100 | | |
| | 30% | 30 | | |
| | 50% | 5 | 10 | 15 |
| HYDROLYZED | 10% | 100 | | |
| GELATIN | 30% | 100 | | |
| | 50% | 100 | | |
| | 70% | 100 | | |

It can be concluded that even disintegrated rejuvenating-nutrients may not dissolve in water in the stomach if there is not sufficient water available for hydration and solubilization. The amount of water in the stomach varies based on the amount of fluids and food consumed. Furthermore, depending on the type of food consumed, a competition between the components of food (protein, carbohydrate, fat, fiber) will compete with rejuvenating-nutrients for available water to start the digestion process. Depending on the condition of the stomach, there may not be sufficient water to dissolve rejuvenating-nutrients for optimal absorption in the small intestine. The result could be inefficient absorption and excretion of rejuvenating-nutrients. In such a case, the consumers have paid for rejuvenating-nutrients they never benefited from.

This invention is further illustrated by the following examples, which are to be regarded as illustrative only, and in no way limit the scope of the invention.

### Example 1

Gelled products were produced according to the teachings of the present invention. The gelled functional snacks were formulated using various rejuvenating-nutrients individually or in combination. The joint-ingredients were added directly to the processor without prehydration. Hydration took place during heat processing. The products were formulated as follows:

| | A | B | C | D | E |
|---|---|---|---|---|---|
| Ingredient | % | % | % | % | % |
| GELATIN | 10.0 | | | | 13.0 |
| MSM | | 1.75 | | | 1.75 |
| CHONDROITIN SULFATE | | | 5.6 | | 5.6 |
| GLUCOSAMINE | | 3.2 | | | 6.4 |
| HYDROLYZED GELATIN | | | | 5.0 | 1.0 |
| FLOJEL 60 STARCH | 6.0 | 14 | 15.0 | 14.0 | |
| CORN SYRUP | 15.0 | 15 | 20.0 | 20.0 | 15.0 |
| SUGAR | 40.0 | 45 | 37.0 | 47.0 | 35.0 |
| CITRIC ACID | 0.5 | 0.8 | 1.5 | 2.0 | 1.2 |
| FLAVORS | 1.0 | 1.0 | 2.0 | 2.0 | 2.0 |
| COLORS | 0.01 | 0.02 | 0.02 | 0.01 | 0.02 |
| WATER | 21.89 | 19.23 | 18.88 | 9.99 | 19.0 |

Resultant functional snacks were packaged in laminated foil pouches, sealed and cooled at room temperature to a set consistency. The organoleptic characteristics of the functional snacks were excellent. The functional snacks melted at about 99° F. when incubated in a commercial incubator.

### Example 2

Sugar-free gelled products were produced according to the teachings of the present invention. The gelled functional snacks were formulated using various rejuvenating-nutrients individually or in combination. The ingredients were prehydrated in water, then added to the processor. The products were formulated as follows:

| | A | B | C | D | E |
|---|---|---|---|---|---|
| Ingredient | % | % | % | % | % |
| GELATIN | 10.0 | | | 9.0 | 6.0 |
| MSM | | | | | 3.5 |
| CHONDROITIN SULFATE | | 5.6 | | 5.6 | 5.6 |
| GLUCOSAMINE | | 7.0 | 6.4 | 6.4 | 6.4 |
| HYDROLYZED GELATIN | | | | 3.0 | 1.0 |
| FLOJEL 60 STARCH | 6.0 | 15.0 | 15.0 | 8.0 | 6.5 |
| MALTITOL | 40.0 | 35.0 | 30.0 | 37.0 | 25.0 |
| GLYCERIN | 15.0 | 15.0 | 14.0 | 10.0 | 25.0 |
| SUCRALOSE | 0.1 | 0.1 | 0.2 | 0.2 | 0.25 |
| FLAVORS | 1.0 | 1.0 | 2.0 | 2.0 | 2.0 |
| CITRIC ACID | 0.5 | 0.8 | 1.5 | 2.0 | 1.6 |
| COLORS | 0.01 | 0.02 | 0.02 | 0.01 | 0.02 |
| WATER | 27.39 | 26.08 | 25.28 | 16.79 | 17.13 |

Resultant functional snacks were packaged in a multiplayer polyester film, sealed and cooled at room temperature to a set consistency. The weight of individual units was ten grams. The organoleptic characteristics of the functional snacks were excellent.

### Example 3

Squeezable viscous products were produced according to the teachings of the present invention. The drinkable functional snacks were formulated using various rejuvenating-nutrients individually or in combination. The joint-ingredients were prehydrated in water, and then added to the processor. The products were formulated as follows:

| | A | B | C | D | E |
|---|---|---|---|---|---|
| Ingredient | % | % | % | % | % |
| GELATIN | 4 | | | | 4.0 |
| XANTHAN GUM | 0.2 | 0.3 | | | 0.2 |
| MSM | | 1.0 | | | 0.35 |
| CHONDROITIN SULFATE | | | 5.6 | | 2.1 |
| GLUCOSAMINE | | 3.2 | | | 2.5 |
| HYDROLYZED GELATIN | 4.0 | | | | |
| THICKENING STARCH | | 2.0 | | 5.0 | |
| HIGH FRUCTOSE | 10.0 | 10.0 | 20.0 | | 10.0 |
| CORN SYRUP | | | | | |
| SUGAR | 20.0 | 25.0 | 20.0 | | 20.0 |
| CITRIC ACID | 0.5 | 0.8 | 0.5 | | 0.5 |
| FLAVORS | 1.0 | 1.0 | 2.0 | | 1.6 |
| COLORS | 0.01 | 0.02 | 0.01 | | 0.01 |
| WATER | 60.29 | 56.68 | 46.89 | | 58.74 |

Twenty-four grams of the resultant functional snacks were packaged in a multiplayer polyester film, sealed and cooled at room temperature to a semi-liquid consistency. The eating quality of the functional snacks was acceptable. Furthermore, the semi-liquid snack was dissolved in an equal amount of water and was consumed as a liquid with good results.

### Example 4

To formula E in example 1, antioxidant blend of Vitamin A, Vitamin C and Vitamin E was added to the liquid product at about 100° F.-120° F., and then the product was packaged in candy like wrapping material.

### Example 5

A dry mix including rejuvenating-nutrients was formulated as follows:

Gelatin 8%, sugar 85%, citric acid 0.14%, Glucosamine 3.5%, Chondroitin powder 3.0%, MSM 0.46%, Ascorbic acid 0.16%, color 0.01%, flavors 0.73%. The dry mix was reconstituted in equal part of boiling water, poured into containers and cooled. After cooling the resultant gel dessert was evaluated for physical and eating attributes. It was concluded that this method of providing rejuvenating-nutrients is very appealing to consumers as well as provides pre-hydrated rejuvenating-nutrients in an easy to use product.

### Example 6

Formulations suitable for dogs with musculoskeletal disorder were developed as follows:

| | A | B | C | D | E |
|---|---|---|---|---|---|
| Ingredient | % | % | % | % | % |
| GELATIN | 15.0 | 12 | | 9.0 | 12.0 |
| MSM | | 0.5 | | | 0.5 |
| CHONDROITIN SULFATE | | | 5.6 | 3.2 | 1.6 |
| GLUCOSAMINE | | 1.6 | 6.4 | 3.0 | 1.0 |
| HYDROLYZED GELATIN | | | | | 1.0 |
| FLOJEL 60 STARCH | 8.0 | 10.0 | 17.0 | 8.0 | 8.0 |
| WHEY SOLIDS | | 10.0 | 15.0 | 20.0 | |
| FLAVORS | 1.0 | 1.5 | 2.0 | 1.0 | 2.0 |
| CITRIC ACID | 0.5 | 0.8 | 1.2 | 2.0 | 0.5 |
| COLORS | 0.01 | 0.02 | 0.02 | 0.01 | 0.02 |
| WATER | 75.49 | 63.58 | 52.78 | 53.79 | 73.38 |

Twenty grams of the resultant chews were served to various puppies and dogs with very good liking by the animals.

What is claimed is:

1. A composition for treating an animal or human comprising:
a gelling agent; and
an additive selected from the group consisting of glucosamine, chondroitin, methyl-sulfonyl-methane, gelatin, hydrolyzed gelatin, collagen, hydrolyzed collagen and mixtures and derivatives thereof.

2. The composition of claim 1, wherein the gelling agent is selected from the group consisting of collagen, gellan gum, carbohydrate gel forming polymers, carrageenan, alginates, guar, xanthan, carboxymethyl cellulose, starch and mixtures thereof.

3. The composition of claim 1, wherein the gelling agent is hydrolyzed.

4. The composition of claim 1, wherein the composition further comprises between about 15% to about 70% water.

5. The composition of claim 1, wherein the composition melts at least about 90° F.

6. The composition of claim 1, wherein the glucosamine includes D-glucosamine at a quantity of about 200 mg to about 2000 mg per dosage.

7. The composition of claim 1, wherein the chondroitin includes chondroitin at a quantity of about 100 mg to about 2000 mg per dosage.

8. The composition of claim 1, wherein the methyl-sulfonyl-methane is included at a quantity of about 100 mg to about 2000 mg per dosage.

9. The composition of claim 1, wherein the gelatin is included about at a quantity of about 200 mg and 5000 mg.

10. The composition of claim 1, wherein the collagen is included at about at a quantity of about 200 mg and 5000 mg.

11. The composition of claim 1, wherein the hydrolyzed collagen is included at about at a quantity of about 100 mg and 2500 mg.

12. The composition of claim 1, wherein the hydrolyzed gelatin is included at about at a quantity of about 100 mg and 2500 mg.

13. The composition of claim 1, further including sweeteners.

14. The composition of claim 9, wherein the sweeteners are selected from the group selected consisting of monosaccharides, disaccharides, sucralose, aspartame, saccharin and combinations thereof.

15. The composition of claim 1, wherein the composition includes a pH between about 2.5 to about 5.5.

16. The composition of claim 15, wherein the composition includes a pH between about 3 to about 4.

17. The composition of claim 1, further including an acidulant.

18. The composition of claim 16, wherein the acidulant is selected from the group consisting of citric acid, tartaric acid, malic acid, ascorbic acid, lactic acid, phosphoric acid, hydrochloric acid and combinations thereof.

19. A method of making a composition for treating a human or animal comprising:
providing a gelling agent;
providing an additive selected from the group consisting of glucosamine, chondroitin, methyl-sulfonyl-methane gelatin, hydrolyzed gelatin, collagen, hydrolyzed collagen and mixtures and derivatives thereof; and
combining the gelling agent and additive to form the composition.

20. The method of claim 19, further comprising hydrating the composition.

21. The method of claim 20, wherein the step of hydrating includes adding water heated to a temperature of between about 110° F. to about 170° F.

22. The method of claim 19, further including pasteurizing the composition.

23. A method of treating a human or animal for musculoskeletal disorders and collagen and elastin deficiencies comprising:
administering to a human or animal a composition comprising a gelling agent; and
an additive selected from the group consisting of glucosamine, chondroitin, methyl-sulfonyl-methane gelatin, hydrolyzed gelatin, collagen, hydrolyzed collagen and mixtures and derivatives thereof.

24. The method of claim 23, wherein the gelling agent is selected from the group consisting of collagen, gellan gum, carbohydrate gel forming polymers, carrageenan, alginates, guar, xanthan, carboxymethyl cellulose, starch and mixtures thereof.

25. The method of claim 23, wherein the gelling agent is hydrolyzed.

26. The method of claim 23, wherein the composition further comprises between about 20% to about 50% water.

27. The method of claim 23, wherein the composition melts at least 90° F.

28. The method of claim 23, wherein the glucosamine is administered at a dosage of about 200 mg to about 2000 mg per dosage.

29. The method of claim 23, wherein the chondroitin is administered at a dosage of about 100 mg to about 2000 mg per dosage.

30. The method of claim 23, wherein the methyl-sulfonyl-methane is administered at about 100 mg to about 2000 mg per dosage.

31. The method of claim 23, wherein the gelatin is included about at a quantity of about 200 mg and 5000 mg.

32. The method of claim 23, wherein the collagen is included at about at a quantity of about 200 mg and 5000 mg.

33. The method of claim 23, wherein the hydrolyzed collagen is included at about at a quantity of about 100 mg and 2500 mg.

34. The method of claim 23, wherein the hydrolyzed gelatin is included at about a quantity of about 100 mg and 2500 mg.

35. The method of claim 23, further including sweeteners.

36. The method of claim 35, wherein the sweeteners are selected from the group selected consisting of monosaccharides, disaccharides, sucralose, aspartame, saccharin and combinations thereof.

37. The method of claim 23, wherein the composition includes a pH between about 2.5 to about 5.5.

38. The method of claim 37, wherein the composition includes a pH between about 3.0 to about 3.6.

39. The method of claim 23, further including an acidulant.

40. A kit comprising a preformed package, the kit comprising:
a gelling agent; and
an additive selected from the group consisting of glucosamine, chondroitin, methyl-sulfonyl-methane gelatin, hydrolyzed gelatin, collagen, hydrolyzed collagen and mixtures and derivatives thereof.

41. The kit of claim 40, wherein the preformed package is hermetically sealed.

42. The kit of claim 40, wherein the composition is a gelled food.
